(19) United States
(12) Patent Application Publication
(10) Pub. No.: US 2026/0224397 A1
(43) Pub. Date: Aug. 6, 2026

(54) MULTI-STATION ROBOTIC OCULAR DRUG DELIVERY DEVICE

(71) Applicant: Alcon Inc., Fribourg (CH)

(72) Inventors: Patrick Terry, Goleta, CA (US); Paul R. Hallen, Colleyville, TX (US); Nanhong LOU, Southlake, TX (US); Lance NOLLER, Yorba Linda, CA (US)

(21) Appl. No.: 19/460,577

(22) Filed: Jan. 27, 2026

Related U.S. Application Data
(60) Provisional application No. 63/749,869, filed on Jan. 27, 2025.

(51) Int. Cl. A61F 9/00; A61G 15/12; A61M 5/42; A61B 34/20; A61B 34/32

(52) U.S. Cl. CPC A61F 9/0017; A61G 15/125; A61M 5/427; A61B 2034/2055; A61B 34/32; A61M 2205/84

(57) ABSTRACT

A drug delivery device includes a conveyor and a docking assembly movable by the conveyor to a plurality of patient stations. The docking assembly includes one or more imaging devices configured to have an eye of the patient in a field of view thereof. The docking assembly includes an injection assembly and a staging assembly including one or more actuators and configured to position the injection assembly relative to the eye of the patient. A controller is configured to receive one or more images from the one or more imaging devices; detect anatomy of the eye of the patient in the one or more images; and activate the one or more actuators to drive a needle mounted to the injection assembly into a placement location on the eye of the patient according to the location of the anatomy. A loader loads injection assemblies into the docking assembly.

### CROSS-REFERENCE TO RELATED APPLICATIONS

This application claims the benefit of U.S. Provisional Application Serial No. 63/749,869 (filed on January 27, 2025), the content of which is incorporated by reference herein in its entirety.

### BACKGROUND

The present disclosure relates generally to instruments used for providing intravitreal injections.

Light received by the eye is focused by the cornea and lens of the eye onto the retina at the back of the eye, which includes the light sensitive cells. The interior of the eye between the lens and the retina is filled with a transparent gel known as the vitreous. Many conditions of the retina are treated by intravitreal injections in which medication is injected into the vitreous. Such conditions include age-related macular degeneration, retinal vein occlusion, diabetic macular edema, diabetic retinopathy, and others. Once diagnosed with a condition requiring intravitreal injections, a patient may continue to require injections periodically.

It would be an advancement in the art to facilitate the administration of intravitreal injections.

### SUMMARY

In certain embodiments, a drug delivery device includes a conveyor. A docking assembly is mounted to the conveyor and configured to receive a portion of a head of a patient. The conveyor is configured to transport the docking assembly to a plurality of stations. The docking assembly includes one or more imaging devices configured to have an eye of the patient in a field of view thereof. The docking assembly further includes an injection assembly configured to receive a drug to be injected into the eye of the patient and a staging assembly including one or more actuators and configured to position the injection assembly relative to the eye of the patient. A controller is coupled to the conveyor, the one or more imaging devices, and the staging assembly. The controller is configured to receive one or more images from the one or more imaging devices; detect a location of anatomy of the eye of the patient in the one or more images; and activate the one or more actuators to drive a needle mounted to the injection assembly into a placement location on the eye of the patient according to the location of the anatomy.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1A is a side view of a multi-station robotic intravitreal drug delivery device in accordance with certain embodiments.

FIG. 1B is a top view of a multi-station robotic intravitreal drug delivery device of FIG. 1A.

FIG. 2A is a top view of a docking assembly of the multi-station portable robotic intravitreal drug delivery device in accordance with certain embodiments.

FIG. 2B is a partial side view of the device of FIG. 2A.

FIGS. 3A to 3D illustrate example injection assemblies in accordance with certain embodiments.

FIG. 4 is a schematic block diagram of a loader for a docking assembly in accordance with certain embodiments.

FIG. 5 is a schematic block diagram of electronic components of a multi-station robotic intravitreal drug delivery device in accordance with certain embodiments.

FIG. 6 is a process flow diagram of a method for preparing for drug delivery using a multi-station robotic intravitreal drug delivery device in accordance with certain embodiments.

FIG. 7 is a process flow diagram of a method for administering an intravitreal injection using a multi-station robotic intravitreal drug delivery device in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

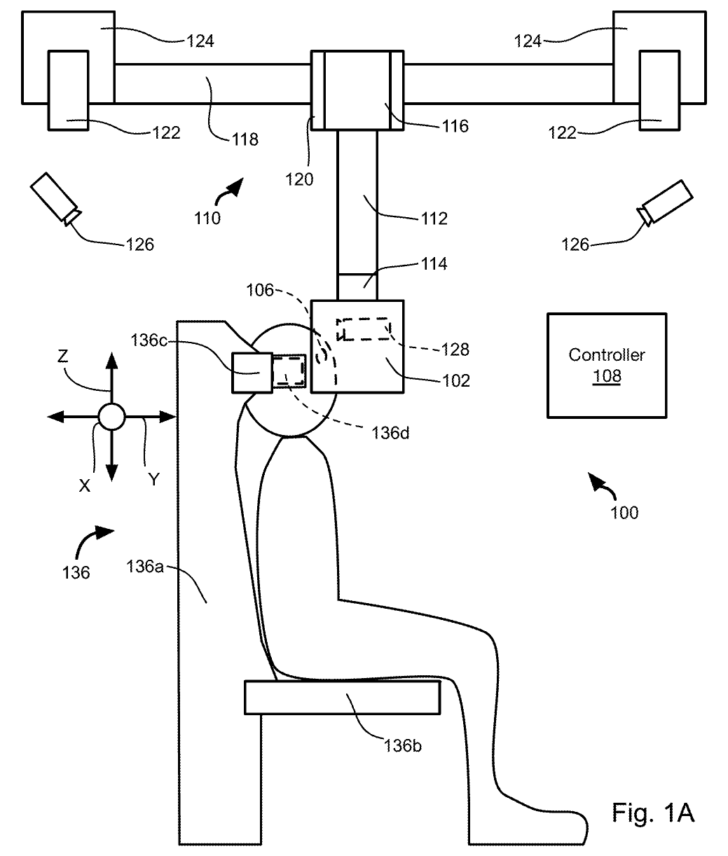

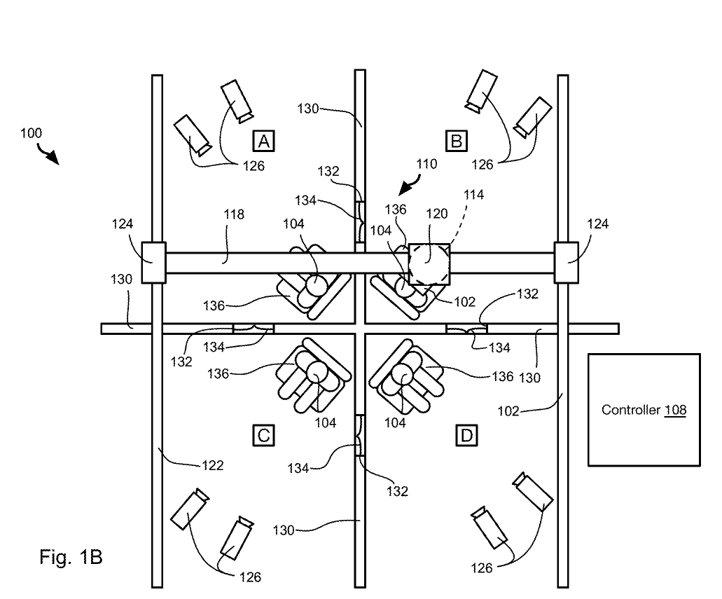

FIG. 1A illustrates an example embodiment of an example multi-station robotic intravitreal drug delivery device 100 (hereinafter “drug delivery device 100”). The drug delivery device 100 includes a docking assembly 102 that receives a portion of the head 104 of a patient and covers one or both eyes 106 of the patient. The docking assembly 102 may include some or all of a forehead rest, cheek rests, temporal rests, chin rest, or rests for engaging other portions of the head 104 of the patient, any of which may be adjustable. The docking assembly 102 may include structures for clamping or otherwise retaining the head 104 of the patient, such as a clamp for pressing pads against temporal regions of the patient’s head 104, a headband encircling the patient’s head 104 and fastened to the docking assembly 102, or other structures. Alternatively, the patient may be relied upon to press the patient’s head against the docking assembly 102 with sufficient stability for the administration of an intravitreal injection as described below.

In the following description, reference is made to intravitreal injections with the understanding that other types of ocular injections to the eye or orbital space of the eye may be performed in a like manner. Other types of ocular injections may include, for example, intracameral injections, subretinal injections, suprachoroidal injections, subconjunctival injections, retro-orbital injections, periorbital injections, and the like.

The docking assembly 102 may be housed separately from a controller 108 and connected to the controller 108 a cable, wireless connection, optical fiber, or other type of connection in order to reduce the weight of the docking assembly 102. The controller 108 includes logic (e.g., a computing device) for controlling actuators and other components of the docking assembly 102 and other actuators of the drug delivery device 100. The controller 108 may include a power supply such as a rechargeable battery or adapter for connecting to an electrical outlet.

The docking assembly 102 is coupled to a conveyor 110 for moving the docking assembly 102 among a plurality of patient stations. The conveyor 110 may be understood with respect to X, Y, and Z direction, where the Z direction is substantially (e.g., within 2 degrees of) parallel to the direction of gravity and the X and Y directions are substantially (e.g., within 2 degrees of) perpendicular to the Z direction and to one another. The conveyor 110 is configured to move the docking assembly 102 in the X, Y, and Z directions as well as at least one rotational degree of freedom, such as rotation about an axis substantially (e.g., within 2 degrees of) parallel to the Z direction.

For example, the docking assembly 102 may be mounted to a support 112 by a rotational actuator 114 that performs rotational movement of the docking assembly 102 relative to the support 112 about the axis substantially parallel to the Z direction. The support 112 extends vertically above the docking assembly 102 in the Z direction and is coupled to a vertical actuator 116. The vertical actuator 116 may actuate the support 112 in the Z direction in order to change the height of the docking assembly 102 in the Z direction to align with the patient’s head 104.

The linear actuator 116 may be mounted to a rail 118 by a linear actuator 120 that induces movement in the Y direction along the rail 118. The rail 118 may have a substantially constant cross-section in planes parallel to the X and Z directions along a majority of the length thereof to facilitate movement of the linear actuator 120 along the rail 118. The linear actuator 120 may include rollers to facilitate movement along the rail 118 as well as a braking mechanism to fix the linear actuator 120 in position on the rail 118.

The rail 118 may be mounted to rails 122 by a linear actuator 124 that induces movement in the X direction along the rails 122. The rails 122 may have a substantially constant cross-section in planes parallel to the Y and Z directions along a majority of the length thereof to facilitate movement of the linear actuator 124 along the rails 122. The linear actuator 124 may include rollers to facilitate movement along the rails 122 as well as braking mechanisms to fix the linear actuator 124 in position on the rails 122.

The controller 108 is coupled to the rotational actuator 114, and linear actuators 116, 120, 124 and controls the activation thereof in order to position the docking assembly 102 adjacent the head 104 of the patient.One or more cameras 126 may be mounted in a space including the conveyor 110 and may each have a region in space likely to be occupied by a patient’s head 104 in a field of view thereof. The controller 108 may receive images from the cameras 126 in order to estimate a three-dimensional position of the patient’s head 104 and activate the conveyor 110 to position the docking assembly 102 at or within a threshold distance of the patient’s head 104. The docking assembly 102 itself may include one or more cameras 128. Images from the one or more cameras 128 may be used by the controller 108 to perform fine adjustments to the position of the docking assembly 102. Alternatively, the docking assembly 102 may incorporate actuators that are controlled to perform fine adjustments of the docking assembly 102 based on one or more images from the one or more cameras 128.

The position of the docking assembly 102 itself may be determined by sensing a kinematic state of the actuators 114,116, 120, 124 using sensors incorporated into the actuators 114, 116, 120, 124 or elsewhere in the conveyor 110. Alternatively or additionally, the position of the docking assembly 102 may also be determined based on images from the one or more cameras 126, 128.

Although cameras 126, 128 are described as being used to estimate the position of the patient’s head 104 and possibly the docking assembly 102, other imaging or sensing modalities may be used such as light detection and ranging (LIDAR), radio detection and ranging (RADAR), ultrasonic sensing, or other type of sensor. The one or more cameras 128 may each be replaced with an optical coherence tomography (OCT) device, scanning laser ophthalmoscope, or other type of ophthalmic imaging device. An OCT device is particularly helpful for tracking the location of a needle during insertion, injection, and withdrawal.

The conveyor 110 in the illustrated embodiment includes a gantry composed of the linear actuators 116, 120, 124. However, other implementations are other possible. For example, the conveyor 110 may be embodied as or include a serial robot arm including one or more rotational and hinge joints, such as commonly available 6 degree of freedom (DOF) serial robotic arms that are available in many different sizes and configurations.

Referring to FIG. 1B, one or more walls 130 may divide a space into two or more stations, such as the illustrated stations A, B, C, and D. Although four stations are shown, there may be 2, 6, 8, 10, or any number of stations of various sizes subject to the space required for patient comfort and a range of motion of the conveyor 110. Any number of stations may be arranged in a cartesian grid arrangement, as wedges surrounding a center point, or other arrangement. In some embodiments, the docking assembly 102 is mounted on a conveyor embodied as a mobile cart that may maneuver itself among multiple rooms of a hospital, clinic, or other facility.

One or more of the walls 130 may defined openings 132 extending downwardly from upper edges thereof and providing clearance for the docking assembly 102 and support 112 to pass therethrough. The openings 132 may have gates 134 secured therein that obstruct the openings 132 in order to provide privacy to patients within each station A, B, C, D. The gates 134 may be made of sheets of flexible polymer, doors on spring-loaded hinges, or other type of gate that may be opened by the conveyor 110 forcing the docking assembly 102 and support 112 therethrough. The gates 134 may include actuators coupled to the controller that open a particular gate to permit the docking assembly 102 and support 112 to pass therethrough and otherwise maintain the gate 134 closed.

In some embodiments, the range of motion of the conveyor 110 in the Z direction is sufficient to lift the docking assembly 102 and support 112 above the walls 130 such that openings 132 and gates 134 are not used.

Each station A, B, C, D may include a patient support 136, such as a seat, bed, or other structure for supporting a patient. The patient support 136 may be fixed relative to each station A, B, C, D to ensure that a patient on the patient support 136 will have the patient’s head 104 within a region that is accessible by the conveyor 110 with the docking assembly 102 positioned to dock with the head 104 of the patient as described below. The location of the patient support 136 may be selected such that a head 104 of a patient supported on the patient support 136 will be in the field of view of the cameras 126.

The patient support 136 may include a vertical support 136a that may be bolted to a ground or otherwise maintained immobile. A seat 136b may be mounted to the vertical support 136a for the patient to set thereon. One or two clamping actuators 136c mount to the vertical support 136a and clamp the patient’s head 104 between pads 136d in order to reduce movement of the patient’s head 104 relative to the vertical support 136a. The clamping actuators 136c may be eliminated in some embodiments in favor of a passive or manually actuated clamp for clamping the patient’s head 104.

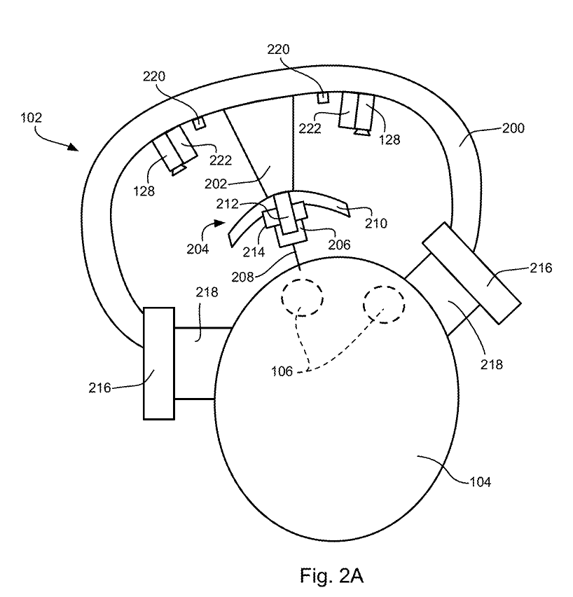

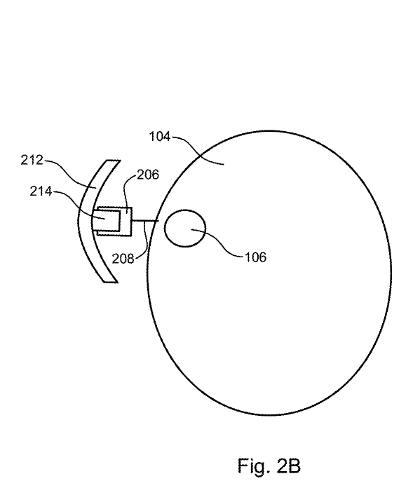

Referring to FIGS. 2A and 2B, the docking assembly 102 includes a frame 200. The frame 200 may be embodied as a track, rail, or other structural member along which components may be fastened at various positions. The frame 200 may include one or more mounting structures 202 mounted thereto. Each mounting structure 202 has a staging assembly 204 mounted thereto. The staging assembly 204 includes one or more actuators that perform fine adjustments, relative to the precision of the conveyor 110, of the position of an injection assembly 206 for injecting a drug into the eye 106 of a patient and for inserting a needle 208 of the injection assembly 206 into the eye 106 of the patient.

In the illustrated embodiment, the staging assembly 204 includes an actuator 210 and an actuator 212 that are oriented substantially (e.g., within 2 degrees of) perpendicular to one another. The actuators 210, 212 may be linear actuators or the illustrated arcuate actuators 210, 212. For example, the actuators 210, 212 may define arcuate actuation paths that are each centered on a remote center of motion. For example, the remote center of motion may lie on the needle 208 or a path followed by the needle 208 when extended by an extension actuator 214 configured to extend and withdraw the needle 208 when performing intravitreal injections. For example, the actuator 210 may be mounted to the mounting structure 202, the actuator 212 may be mounted to the actuator 210 and be actuated thereby along a first actuate path. The extension actuator 214 may be mounted to the actuator and may be actuated thereby along a second arcuate path that has the same remote center of motion as the first arcuate path, e.g., within 1 mm, .01 mm, or 1 micron. The injection assembly 206 may be mounted to the extension actuator 214 with the needle 208, or a line extending along the center of the lumen of the needle 208 lying on the remote center of motion, e.g., within 1 mm, .01 mm, or 1 micron.

One or more clamping actuators 216 may be mounted to the frame 200. The clamping actuators 216 are configured to extend one or more pads 218 into engagement with the head 104 of the patient in order to reduce movement of the head of the patient relative to the docking assembly 102. For example, there may be two pads 218 with one pad or both pads being coupled to clamping actuators 216 for decreasing the distance between the two pads 218 in order to clamp the head 104 of the patient.

In practice, the docking assembly 102 is positioned relative to the head 104 of the patient using the conveyor 110 and images from the one or more cameras 126. One or more images from the one or more cameras 128 of the docking assembly 102 may be used to determine the relative position of the eye 106 of the patient and perform fine adjustments using the conveyor 110 based on the position. Once in position, the clamping actuators 216 may be activated to bring the pads 218 into engagement with the head 104 of the patient. Note that the position of the pads 218 may be asymmetric relative to the head 104 of the patient since the same docking assembly 102 may be used in two different positions to perform intravitreal injections on the right and left eyes 106 of the patient. The actuation of the clamping actuators 214 may be guided by images from the one or more cameras 128. For example, the clamping actuators 214 may be used to adjust the relative positions of the docking assembly 102 and the patient’s head 104.

The goal of positioning of the docking assembly 102 may be to position the needle 208 on a line that intersects a point on the eye 106 of the patient at a prescribed position and angle, or within a tolerance of such a position and angle that is within the range of motion provided by the staging assembly 206. For example, the prescribed position may be between 3 and 3.5 millimeters from the limbus for an aphakic eye and between 3.5 and 4 millimeters from the limbus for a phakic/pesudophakic eye. The prescribed angle may be determined as known in the art of intravitreal injections and may be selected such that upon insertion of the needle, the needle avoids contact with the lens and retina while placing medication near the retina or area of the retina to be treated.

The docking assembly 102 may include one or more electronic components in addition to the one or more cameras 128. The docking assembly 102 may include one or more fixation targets 220. Each fixation target 220 may be embodied as a static image, light source, screen for displaying a fixation target, or other device. A separate fixation target 220 may be provided for each eye 106 or a single fixation target 220 may used for both right and left eyes 106. Alternatively, a single fixation target 220 may be mounted at different positions on the frame 200 for different eyes 106. In some embodiments a single fixation target 220 is centrally located to be used for both eyes 106, i.e., patient may direct each eye 106 toward the nose of the patient in order to expose the sclera for receiving an injection. Alternatively, a single screen implementing the fixation target 220 may display a fixation target at a different location for each eye 106. The location of the fixation target 220 may be adjusted using software executed by the controller 108 or by an observer in order to induce the patient to position the eye 106 at a desired angle.

The docking assembly 102 may include one or more intraocular pressure (IOP) sensors 222. The IOP sensor 222 may be a contact or non-contact sensor and may be used during intravitreal injection to ensure that the IOP of the patient’s eye 106 does not increase to unsafe levels. There may be separate IOP sensors 222 for each eye or a single IOP sensor 222 may be mounted at different positions on the frame 200 in order to measure the IOP of each eye 106.

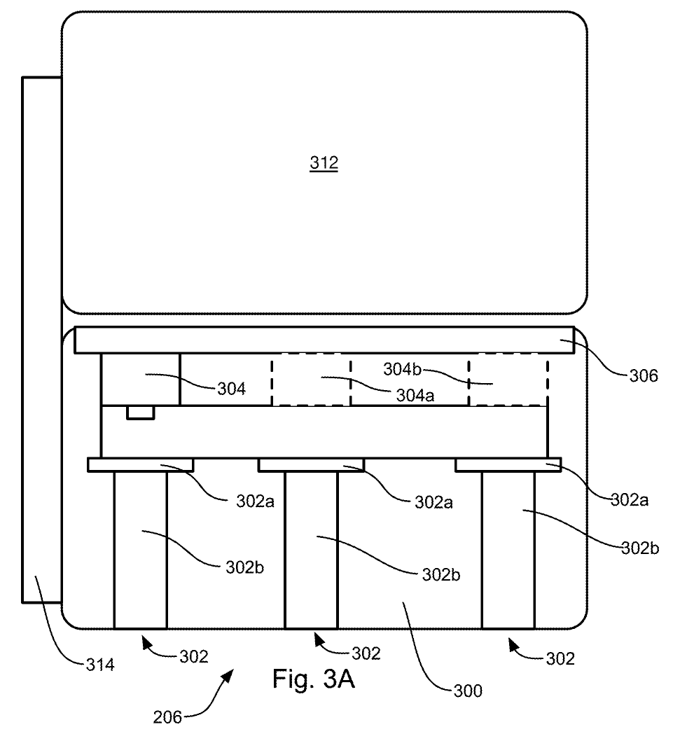

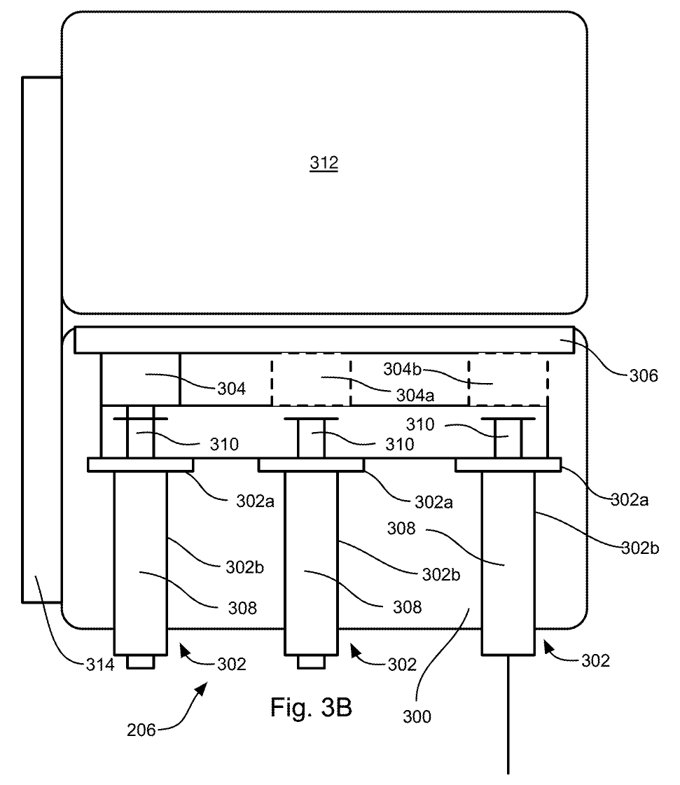

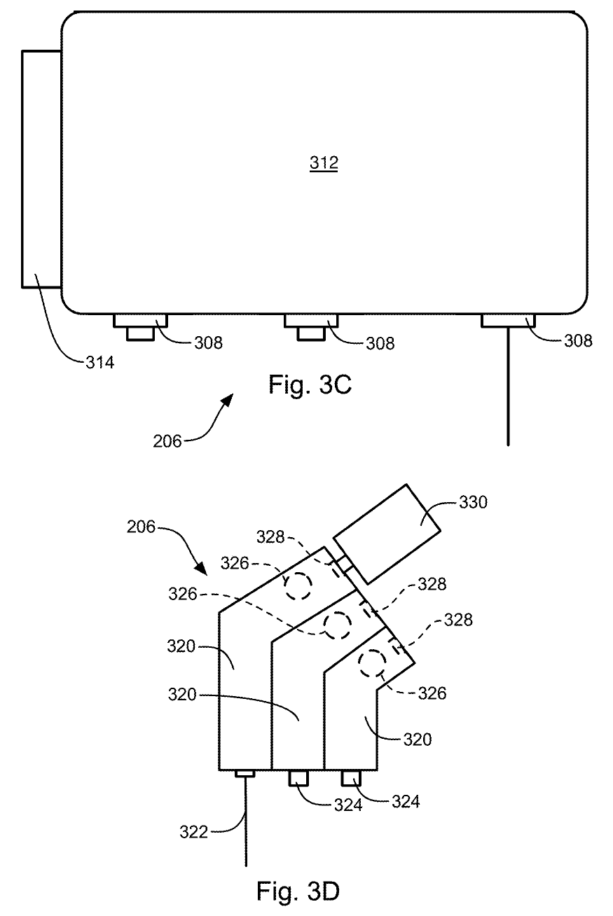

Referring to FIGS. 3A to 3C, the injection assembly 206 may have some or all of the attributes and/or functionalities described below. The injection assembly 206 may include a tray 300 defining one or more recesses 302 for receiving syringes, such as three recesses 302 for receiving syringes containing an anesthetic, a disinfectant, and a drug to be delivered by intravitreal injection. For example, each recess 302 may include a groove 302a for receiving a flange of a syringe and a recess 302b connected to the groove 302a for receiving the barrel of the syringe.

A plunger actuator 304 is positioned to depress the plunger 310 of syringes 308 positioned within the recesses 302. In some embodiment, a single plunger actuator 304 is used and is moved by a positioning actuator 306 between the illustrated position and two other positions 304a, 304b in order to depress the plunger 310 of syringes positioned in each of the recesses 302. In other embodiments, a separate plunger actuator 304 is provided to depress the plunger 310 of a syringe 308 positioned in each recess 302.

Syringes 308 may be retained within the recesses 302 by means of a lid 312 or other retention structure. The lid 312 may be coupled to a lid actuator 314 that can be moved into the open position of FIGS. 3A and 3B and into the closed position of FIG. 3C in which the lid 312 is positioned over the syringes 308 positioned within the recesses 302. The lid 312 may be flat or may include recesses that receive portions of the syringes 308 when the lid 312 is in the closed position over the tray 300. For example, the lid 312 may include recesses 302 similarly to the tray 300, each recess 302 including a groove 302a for receiving a flange of a syringe 308 and a recess 302b for receiving the barrel of a syringe 308.

FIG. 3D illustrates an alternative implementation for the injection assembly 206. In the illustrated embodiment, the injection assembly 206 includes one or more reservoirs 320, such as the illustrated reservoirs 320 for containing a drug to be administered, an anesthetic, and a disinfectant. The reservoirs 320 may be separate members or joined together by fasteners, placement in a common housing, or co-molding. Each reservoir 320 may have an outlet formed thereon or secured thereto, such as in the form of a hypodermic needle 322 or nozzle 324 for dispensing fluid.

Each reservoir 320 may have a pump 326 associated therewith. The pump 326 of each reservoir 320 may be used to force fluid out of the outlet of the reservoir 320. The pump 326 may be replaced with other propulsion sources. For example, pressurized fluid may be forced into a reservoir 320 and engage a piston or bladder in order to force fluid out of the reservoir 320.

Each reservoir 320 may have an inlet 328 for filling the reservoir 320. The inlet 328 may be coupled to a vial 330 or syringe containing fluid to be loaded into the reservoir 320. The fluid may be forced into the reservoir 320 using a syringe or other pressure source. Alternatively, the pump 326 of a reservoir 320 may be activated in order to draw fluid out of a vial 330 through the inlet 328 of the reservoir 320. In other implementations, fluid may be drawn through the inlet 328 or outlet of a reservoir 320 and into a bladder within a reservoir 320 by reducing pressure in the reservoir 320 around the bladder, such as through a port for coupling to a pneumatic pressure source. In some embodiments, the reservoirs 320 may be large enough to store multiple doses. In such embodiments, the injection assembly 206 may include refrigeration to reduce degradation of a drug to be injected.

The inlet 328 may be include a one-way valve, self-sealing polymer defining a hole for receiving a needle, removable cap, or other closure mechanism. In some embodiments, the injection assembly 206 is a disposable cartridge that is pre-loaded with fluid such that an inlet 328 is omitted. For example, the reservoirs 320 may be filled through the outlet thereof at the time of manufacture.

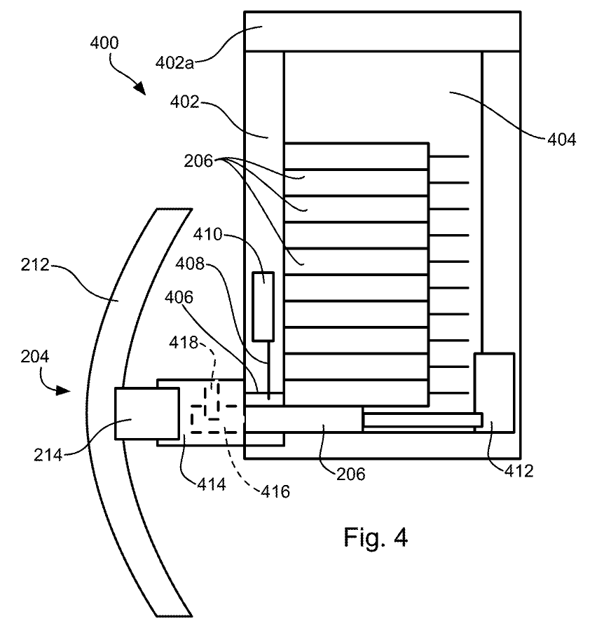

Referring to FIG. 4, the staging assembly 204 may interface with a loader 400 to load an injection assembly 206 into the staging assembly 204. In some implementations, one of the stations A, B, C, D may be used to house the loader 400. The loader 400 may include a housing 402 storing a stack of injection assemblies 206. The housing 402 may be insulated and may be refrigerated to avoid degradation of drugs loaded into the injection assemblies 206. The housing 402 may include a lid 402a positioned at a top or side thereof and which may be opened to provide access to a cavity 404 into which the injection assemblies 206 may be loaded.

The housing 402 may define an opening 406 at a bottom of the cavity 404 through which injection assemblies 206 may be forced out of the cavity 404. The opening 406 may have a gate 408 positioned over the opening 406 to isolate the injection assemblies from external heat and air. The gate 408 may be actuated by an actuator 410 to temporarily open the gate 408 away to permit an injection assembly to be forced out of the opening 406 and into the staging assembly 204. An actuator 412 may be mounted to the housing 402 and, when activated, force a bottommost injection assembly 206 in the cavity 404 out of the opening 406.

The staging assembly 204 may include a housing 414 for receiving the injection assembly 206. The housing 414 may be mounted to the extension actuator 214 and define a cavity 416 for receiving the injection assembly 206. A latch 418 mounted to the housing 414 may engaged the injection assembly 206 to hold the injection assembly 206 firmly in place and resist removal thereof. The latch 418 may be a passive or actuated latching mechanism. The cavity 416 may include electrical contacts through which power is supplied to actuators 304, 306 or pumps 326 of the injection assembly 206. Removal of the injection assembly 206 may be performed by a human operator or one or more actuators disengaging the latch 418 and drawing the injection assembly out of the cavity 416. In some embodiments, a disposal station may be provided for removing the injection assemblies 206 from the housing cavity 416 after use.

In operation, a human operator or algorithm may generate a schedule of patients and assign each patient a station A, B, C, D. An injection assembly 206 loaded with the drug to be injected to each eye of each patient is loaded into the loader 400 in an order corresponding to the schedule: the lower in the stack within the loader 400 the earlier in the schedule. The docking assembly 102 may be brought by the conveyor 110 to the loader 400 with the staging assembly 204 positioned over the opening 406. Injection assemblies 206 may be loaded into the staging assembly 204, transported by conveyor 110 to the station A, B, C, D indicated in the schedule and administered to the patient. The injection assembly 206 may then be removed by a human operator or one or more actuators and the process repeated for the next patient, or for the other eye of the same patient, to be treated according to a schedule.

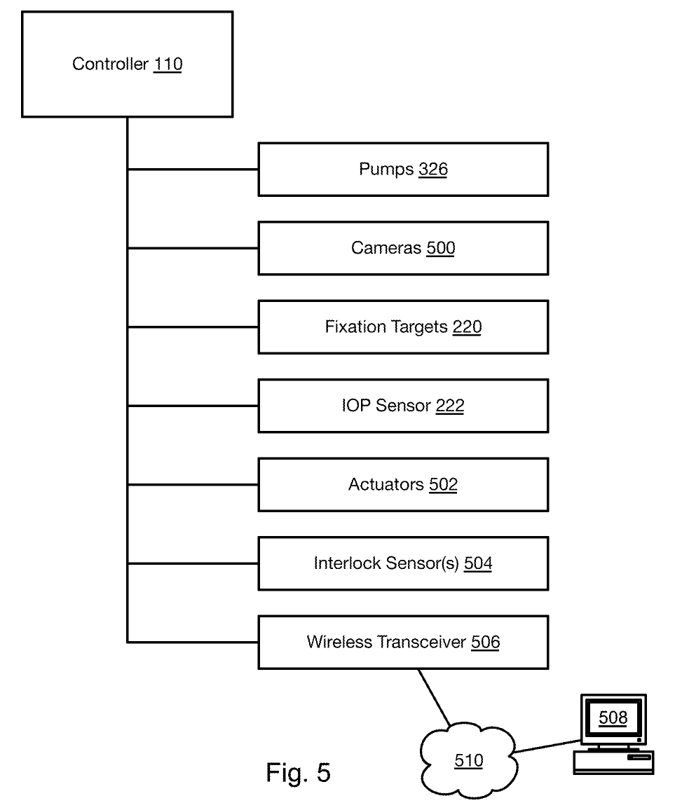

Referring to FIG. 5, the controller 108 is coupled to some or all the one or more pumps 326 of an injection assembly, one or more cameras 500 (e.g., cameras 126, 128), one or more fixation targets 220, the one or more IOP sensors 222, and actuators 502 including some or all of the actuators of the injection assembly 206, conveyor 110, staging assembly 204, and loader 400.

The controller 108 is configured to receive images from the one or more cameras 500 and IOP readings from the one or more IOP sensors 222. The controller 108 may receive feedback from the one or more pumps 326, such as measurements of pressure at the input and/or output of each pump of the one or more pumps 326, current drawn by each pump of the one or more pumps 326, or other information. In some embodiments, feedback from the one or more pumps 326 may be used to obtain an estimated IOP reading and the one or more IOP sensors 222 may be omitted. The controller 108 may possibly receive feedback regarding the state of some or all of the actuators 502 (e.g., current angular or translational position, velocity, and/or acceleration).

The controller 108 may be coupled to one or more interlock sensors 504 that detect a state of the drug delivery device 100 relative to the head 104 of the patient. For example, interlock sensors 504 may sense whether a patient’s head 104 is clamped between the pads 218, whether the injection assembly 206 is properly mounted to the staging assembly 204, or that any of the components described herein is positioned and functioning properly.

The controller 108 may be coupled to a wireless transceiver 506. The operation of the controller 108 may be subject to authorization and instructions received from a computing device 508 over a network 510 by way of the wireless transceiver 506. The controller 108 may authenticate a user of the computing device 508 prior to permitting control using the computing device 508. In some embodiments, the drug delivery device 100 is used in a clinic or hospital in which medical supervision may be provided in-person or by a locally connected interface such that the wireless transceiver 506 may be omitted.

In some embodiments, the observer is remote and may interact with the patient during a procedure, such as by means of an output device such as a screen, speakers, or other device incorporated into the docking assembly 102. Instructions to the patient may be output from the output device either automatically or in response to instructions from the remote observer. The patient may interact with the remote observer using an input device incorporated into the docking assembly 102, such as the one or more cameras 126, a microphone, a touch screen, pointing device, a keyboard, or other input device.

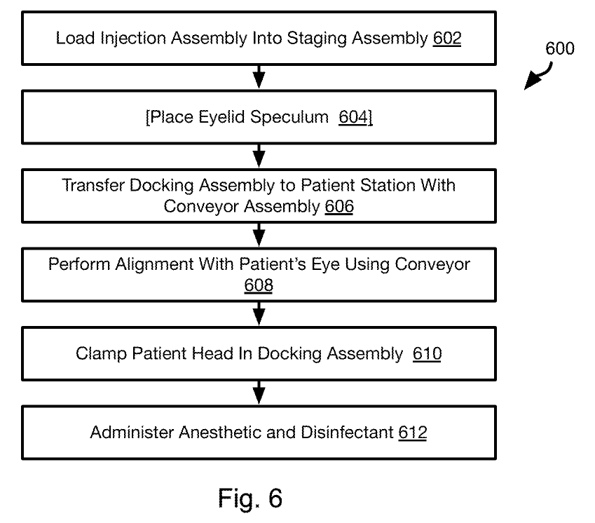

FIG. 6 is a process flow diagram of a method 600 for preparing for drug delivery using the drug delivery device 100. The method 600 includes loading, at step 602, fluid into the injection assembly 206, including the drug to be delivered, an anesthetic, and a disinfectant as described above with respect to FIGS. 3A to 3D. As an alternative, a disposable injection assembly 206 may be provided that is already loaded with fluid such that step 602 is not performed by the patient. Step 602 may include receiving the injection assembly 206 from a loader 400 as described above.

The method 600 may include placing, at step 608, by the patient, a medical professional, or an actuated device may place, a speculum in an eye 106 of the patient to be treated. In other embodiments, the patient is relied upon to maintain the eyelid out of the way such that a speculum is not used. In still other embodiments, an actuated speculum is incorporated into the staging assembly 204 and withdraws the eyelid automatically.

The method 600 includes transferring, at step 606, the docking assembly 102 to the station A, B, C, D where a patient is located and performing, a step 608, alignment of the docking assembly 102 with the head 104 and eye 106 of the patient using the conveyor 110. The alignment of step 608 may be sufficient to place the staging assembly 204 within a tolerance of a desired position and alignment with respect to the eye 106 to be treated and the staging assembly 204 may then perform fine adjustments within the tolerance. For example, alignment performed with the conveyor 110 may be performed to within a tolerance that is less than or equal to a range of motion of the staging assembly 204, such as at less than or equal to half the range of motion of the staging assembly 204 along the X, Y, and Z directions. The alignment of step 608 may be performed using localization of the eye 106 of the patient using one or more cameras 126 and possibly one or more cameras 128.

The method 600 may include clamping, at step 610, a patient’s head 104 in the docking assembly 102, such as by activating actuators 216 to bring pads 218 into engagement with the patient’s head. Activation of the actuators 216 may be performed using feedback from the one or more cameras 128 to urge the patient’s eye 106 into a prescribed position and orientation relative to the staging assembly 204, or at least avoid substantially (e.g., less than 1 mm or less than 2 degrees) disturbing alignment achieved at step 608.

The method 600 may include administering, at step 612, an anesthetic and a disinfectant, such as from the injection assembly 206. Step 612 may be an automated step in which each of the anesthetic and disinfectant is dispensed by depressing a plunger of a syringe using a plunger actuator 304 or activating a pump 326. The outlets of the syringes 308 or reservoirs 320 used to dispense the anesthetic and disinfectant may be placed close to the eye 106 being treated, e.g., within 1 millimeter, or in contact with the eye 106. Alternatively, fluid may be sprayed at step 612 such that such proximity is not required. In some embodiments, step 612 is performed manually by a patient prior to performing the method 600.

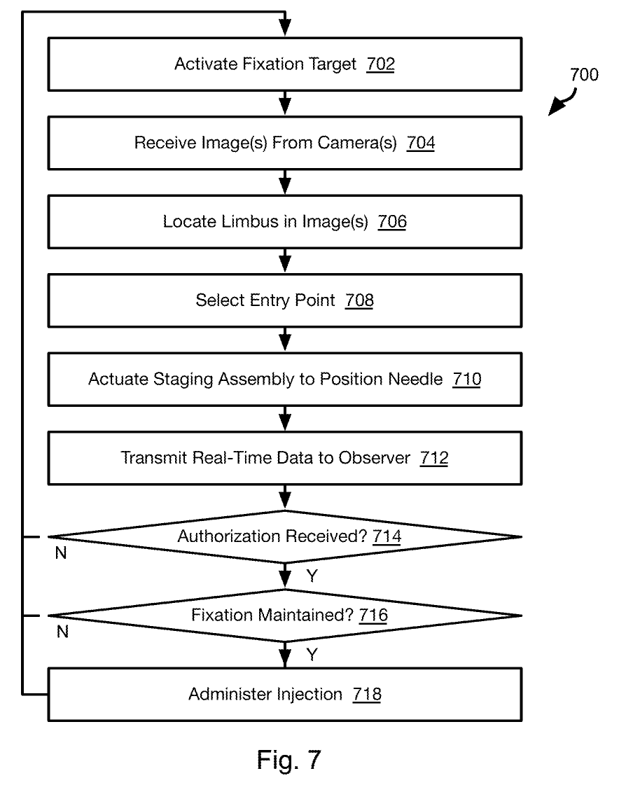

FIG. 7 is a process flow diagram of a method 700 for administering an intravitreal injection using the drug delivery device 100. The method 700 may be performed following performance of the method 600 by the controller 108 activating components of the drug delivery device 100. The method 700 may be performed after waiting for a prescribed time following performance of step 612 to provide time for anesthetic and disinfectant to work.

The method 700 includes activating, at step 702, a fixation target 220. Activating the fixation target 220 may include activating a light, e.g., light emitting diode, displaying an image on the screen, or otherwise providing a visual indicator that is visible to the eye to be treated. Where the fixation target is a static visible structure, step 702 may be omitted. Step 702 may include outputting visual or audible instructions to the patient to fixate on the fixation target 220.

The method 700 includes receiving, at step 704, one or more images from the one or more cameras 128 having the eye 106 to be treated in the field of view thereof. The images received at step 704 may be received in the form of one or more video feeds from the one or more cameras 128.

The method 700 includes locating, at step 706, the limbus of the eye 106 represented in the one or more images, i.e., the boundary between the cornea and the sclera. Step 706 may be performed by registering one or more labeled reference image with respect to the one or more images, the labeled reference image including a label of the limbus. Step 706 may be performed using a machine learning model trained to perform the task, machine vision algorithm, or other approach. For other types of ocular injections, anatomy of the eye, including the orbital space of the eye, other than the limbus may be located at step 706 in order to facilitate placement of the ocular injection.

The method 700 may include selecting, at step 708, an entry point relative to the limbus, or other anatomy identified at step 706. For example, any point within a band of permitted offsets from the limbus, such as between 3 and 3.5 millimeters for an aphakic eye and between 3.5 and 4 millimeters for a phakic eye. The angular position of the entry point about the optical axis of the eye 106 to be treated may be selected as a position that is not obscured by an eyelid of the patient. Other items of anatomy may be identified at step 706, such as the lens and the retina. In some embodiments, the controller 108 generates a three-dimensional model of the eye 106 and uses the model to precisely select the entry point and orientation for the needle in order to avoid damaging ocular tissue, such as the lens, retina, or other items of anatomy. Note that in some applications, the needle will be relatively short (e.g., about 8 mm) such that the angle and depth are not critical for avoiding harm to ocular tissue. In other applications, the needle is used to provide a sub-retinal injection such that angle and depth of penetration are important.

The method 700 may include actuating, at step 710, the staging assembly 204 such that the needle 208 of the injection assembly 206 is pointed at the entry point along the actuation direction of the actuator extension actuator 214. In some embodiments, only translational positioning is performed. However, in others, step 710 may include changing an orientation of the injection assembly 206 such that a needle of the injection assembly 206 is oriented at a desired angle relative to the normal vector of the selected entry point. The desired angle is as known in the art of intravitreal injections and may be selected such that upon insertion of the needle, the needle avoids contact with the lens and retina while placing medication near the retina or area of the retina to be treated. In some embodiments, if the range of motion of the staging assembly 204 is not sufficient to position the needle pointed at the selected entry point, the method 700 may end or the user may be instructed how to adjust the patient’s head 104 relative to the docking assembly 102 to make proper positioning possible.

Step 710 may be performed along with one or more additional iterations of some or all of steps 704, 706, 708 to account for movement of the eye 106 to be treated. Likewise, step 710 may include identifying a representation of the needle 208 in the one or more images received from the one or more cameras 128 and using the representation as feedback to guide positioning of the needle relative to the selected entry point.

The method 700 may include transmitting, at step 712, real time data to an observer, such as to the computing device 508 of an authenticated medical professional. The real time data may include images from the one or more cameras 128, such as by forwarding a video feed from the one or more cameras 128. The real time data may include a representation of the selected entry point from step 708 and a location and orientation of the needle, such as in the form of annotations to images from the one or more cameras 128. The real time data may include reports of successful application of anesthetic and disinfectant, which may include an amount of each applied. The real time data may include outputs of one or more interlock sensors 504 indicating whether the patient is properly positioned and components of the drug delivery device are locked in place and functioning correctly.

The method 700 may include performing one or more verifications prior to administering, at step 718, an intravitreal injection. In some embodiments, some or all of steps 702-712 may be repeated until the verifications are successful or the method 700 is ended by the patient or the observer. The verifications may include verifying, at step 714, that authorization was received from the observer and verifying, at step 716, that fixation of the eye 106 to be treated has been maintained. For example, step 716 may include verifying, using a video feed from the one or more cameras 128, that movement of the eye 106 to be treated is below a maximum threshold, e.g., less than 1 degree, 0.5 degrees, or 0.1 degrees. Step 716 may include verifying that fixation (e.g., movement less than the maximum threshold) was maintained for at least a minimum time period, e.g., from 1 to 3 seconds. Other verifications may include verifying the identity of the patient, such as by verifying that an iris or retina in one or more images from the one or more cameras 128 matches one or more reference images of an iris and/or retina or representation thereof accessed by the controller. In some embodiments, an explicit instruction must be received from the patient to verify that step 718 can be performed, such as in the form of pressing or releasing a button, a verbal command, or visible gesture detected by a camera coupled to the controller 108.

Administering the intravitreal injection at step 718 may include activating the extension actuator 214 to drive the needle 208 into the eye 106 to be treated and activating a plunger actuator 304 or pump 326 to force fluid through the needle and into the eye 106. Step 718 may be performed simultaneously with one or more actions that may include verifying continued authorization by the observer. For example, an observer may continue to receive a video feed from the one or more cameras 128. The observer may hold a button throughout the procedure and release the button in the event that the observer believes that the intravitreal injection should be aborted. In response to receiving notification of release of the button, the controller 108 may abort the intravitreal injection. This approach to continued authorization is exemplary only and other approaches may be used, such as the observer pressing a button or interacting with another user interface element to invoke transmission of an instruction to the controller 108 to abort the intravitreal injection by the controller 108. Step 718 may likewise be aborted in response to an input from the patient in the form of pressing or releasing a button, a verbal command, or visible gesture detected by a camera coupled to the controller 108.

Likewise, fixation may continue to be evaluated as described above with respect to step 716. In the event that fixation is not maintained, the intravitreal injection may be aborted. The IOP within the eye 106 to be treated may be evaluated using outputs of the IOP sensor 222. In the event that the IOP rises fastener than a prescribed rate or above a prescribed pressure, the intravitreal injection may be aborted or the rate of drug delivery may be slowed. In some embodiments, the rate of injection of fluid is regulated based on feedback regarding IOP in order to maintain pressure within the eye 106 below a threshold or a pressure-vs-time curve, with time being measured from when fluid injection began.

In some embodiments, the staging assembly 204 may be activated during step 718 in order to at least partially compensate for movement of the eye 106 to be treated relative to the needle. For example, the staging assembly 204 may include one or more strain sensors sensing strain on the needle in one or more dimensions. The controller 108 may activate one or more of the actuators 210, 212, 214 to reduce the amount of strain sensed by the strain sensors. Step 718 may be aborted in response to movement of the eye 106 to be treated exceeding the range of motion and/or speed of movement required for the staging assembly 204 to compensate for the movement of the eye 106.

Aborting the intravitreal injection may include causing the extension actuator 214 to withdraw the needle of the injection assembly 206 from the eye 106 to be treated to a safe distance from the eye 106 to be treated. Once aborted, the controller 108 may require repetition of the methods 600 and 700. Alternatively, once aborted, the controller 108 may disable further intravitreal injections and require the patient to visit a medical professional for the intravitreal injection.

Step 718 may include monitoring the amount of drug delivered, e.g., amount by which a plunger of a syringe was depressed or amount of pumping performed by the pump 326. Accordingly, an amount of drug that remains to be administered may be determined by the controller 108 and provided to an observer or used by the controller 108 to control the amount of drug delivered in a subsequent iteration of the method 700.

Once the methods 600 and 700 are performed for one eye to be treated, the clamping actuators 216 may withdraw the pads 218 to release the head 104 of the patient. The conveyor 110 may then move the docking assembly 102 away from the head 104 of the patient and the injection assembly 206 may be removed from the staging assembly 204 by a human operator or automated disposal station. The conveyor 110 may move the docking assembly 102 to the loader 400 to receive a new injection assembly 206 and move to a different station A, B,C, D to treat a different patient or back to the same station A, B, C, D to treat a different eye 106 of the same patient.

In some embodiments, the docking assembly 102 may include two staging assemblies 204 and two corresponding injection assemblies 206. The methods 600 and 700 may be performed for each eye 106 of the same patient in series, in parallel, or in an interleaved manner. For example, administration of disinfectant and anesthetic may be performed for both eyes 106 in parallel whereas fixation and injection (e.g., steps 714-718) may be performed in series.

The methods 600 and 700 are exemplary only and may be modified to perform additional steps or ophthalmic treatments. For example, although a drug to be delivered, an anesthetic, and a disinfectant are mentioned above, other fluids may also be used to treat the eye 106 either before or after an injection. For example, some or all of a cooling spray (e.g., saline), anti-inflammation cream or spray, anti-bleeding solution may also be loaded into the injection assembly 206 and applied using the injection assembly 206. In some embodiments, the staging assembly 204 may include one or more actuators that are activated by the controller to press a pad (e.g., cotton or other absorbent material) against an injection site following injection in order to reduce bleeding. Likewise, an actuated pad incorporated into the staging assembly 204 may be pressed against the eye 106 during injection to resist movement of the eye.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. §112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

1. A drug delivery device comprising:
a conveyor;
a docking assembly configured to receive a portion of a head of a patient, the docking assembly being coupled to the conveyor and the conveyor configured to transport the docking assembly to a plurality of stations, the docking assembly comprising:
one or more imaging devices configured to have an eye of the patient in a field of view thereof;
an injection assembly configured to receive a drug to be injected into the eye of the patient; and
a staging assembly including one or more actuators and configured to position the injection assembly relative to the eye of the patient; and
a controller coupled to the conveyor, the one or more imaging devices, and the staging assembly, the controller configured to:
receive one or more images from the one or more imaging devices;
detect a location of anatomy of the eye of the patient in the one or more images; and
activate the one or more actuators to drive a needle mounted to the injection assembly into a placement location on the eye of the patient according to the location of the anatomy of the eye.

2. The drug delivery device of claim 1, wherein each station of the plurality of stations is sized to receive a patient.

3. The drug delivery device of claim 1, wherein the conveyor comprises one or more linear actuators and at least one rotational actuator.

4. The drug delivery device of claim 1, wherein the conveyor is a gantry.

5. The drug delivery device of claim 1, wherein the plurality of stations are defined by one or more walls.

6. The drug delivery device of claim 5, wherein the one or more walls define one or more openings sized to permit the docking assembly to pass therethrough.

7. The drug delivery device of claim 6, further comprising a gate positioned in each opening of the one or more openings and configured to remain closed when the docking assembly is not passing therethrough.

8. The drug delivery device of claim 1, wherein the docking assembly comprises one or more clamping actuators configured to secure the head of the patient relative to the docking assembly.

9. The drug delivery device of claim 1, wherein the docking assembly further comprises a fixation target, the controller being further configured to activate the one or more actuators to drive the needle into the placement location in response to verifying fixation of the eye of the patient on the fixation target.

10. The drug delivery device of claim 1, further comprising a loader, the controller configured to activate the conveyor to transfer the docking assembly to the loader and activate the loader to load the injection assembly into the staging assembly.

11. A method for drug delivery comprising:
activating, by a controller, a conveyor to transport a docking assembly to a station having a patient positioned therein;
receiving, by the controller, one or more images from one or more imaging devices mounted to the docking assembly;
detect a location of anatomy of an eye of the patient in the one or more images;
activating, by the controller, one or more actuators of a staging assembly mounted to the docking assembly to align an injection assembly mounted to the docking assembly relative to the location of the anatomy of the eye; and
activate, by the controller, the one or more actuators to drive a needle mounted to the injection assembly into a placement location on the eye of the patient according to the location of the anatomy of the eye.

12. The method of claim 11, further comprising aligning, by controller, by activating the conveyor, the docking assembly with respect to the eye of the patient.

13. The method of claim 11, wherein the conveyor comprises one or more linear actuators and at least one rotational actuator.

14. The method of claim 11, wherein the conveyor is a gantry.

15. The method of claim 11, wherein the station is one of a plurality of stations, the plurality of stations being defined by one or more walls.

16. The method of claim 15, further comprising activating, by the controller, the conveyor to move the docking assembly through one or more openings defined by the one or more walls.

17. The method of claim 16, further comprising activating, by the controller, the conveyor to move the docking assembly through one or more gates covering the one or more openings defined by the one or more walls.

18. The method of claim 11, further comprising activating, by the controller, one or more clamping actuators to secure a head of the patient relative to the docking assembly.

19. The method of claim 11, wherein the docking assembly further comprises a fixation target, the method further comprising activating, by the controller, the one or more actuators to drive the needle into the placement location in response to verifying fixation of the eye of the patient on the fixation target.

20. The method of claim 11, further comprising a loader, the method further comprising:
activating, by the controller, the conveyor to transfer the docking assembly to the loader; and
activating, by the controller, the loader to load the injection assembly into the staging assembly.
